SEQUENCE LISTING (2 sequences)

SEQ ID NO: 1 - DNA, 40 bp, Artificial Sequence
  primer for yeast two-hybrid
gcaggaaacagctatgaccatacagtcagcggccgccacc

SEQ ID NO: 2 - DNA, 39 bp, Artificial Sequence
  primer for yeast two hybrid
acggccagtcgcgtggagtgttatgtcatgcggccgcta

(19) United States
(12) Patent Application Publication
(10) Pub. No.: US 2002/0098514 A1
(43) Pub. Date: Jul. 25, 2002

(54) Protein-protein interactions

(75) Inventor: Karen Heichman, Salt Lake City, UT (US)

Correspondence Address: ROTHWELL, FIGG, ERNST & MANBECK, P.C., 555 13TH STREET, N.W., SUITE 701, EAST TOWER, WASHINGTON, DC, 20004, US

(73) Assignee: MYRIAD GENETICS, INC., Salt Lake City, UT (US)

(21) Appl. No.: 09/861,664

(22) Filed: May 22, 2001

Related U.S. Application Data
(60) Provisional application No. 60/205,571, filed on May 22, 2000.

(51) Int. Cl. G01N 33/53; C07K 1/00; C07K 14/00; C07K 17/00

(52) U.S. Cl. 435/7.1; 530/350

(57) ABSTRACT

The present invention relates to the discovery of novel protein-protein interactions that are involved in mammalian physiological pathways, including physiological disorders or diseases. Examples of physiological disorders and diseases include non-insulin dependent diabetes mellitus (NIDDM), neurodegenerative disorders, such as Alzheimer's Disease (AD), and the like. Thus, the present invention is directed to complexes of these proteins and/or their fragments, antibodies to the complexes, diagnosis of physiological generative disorders (including diagnosis of a predisposition to and diagnosis of the existence of the disorder), drug screening for agents which modulate the interaction of proteins described herein, and identification of additional proteins in the pathway common to the proteins described herein.

### CROSS-REFERENCE TO RELATED APPLICATION

The present application is related to U.S. provisional patent application Ser. No. 60/205,571 filed May 22, 2000, incorporated herein by reference.

### BACKGROUND OF THE INVENTION

The present invention relates to the discovery of novel protein-protein interactions that are involved in mammalian physiological pathways, including physiological disorders or diseases. Examples of physiological disorders and diseases include non-insulin dependent diabetes mellitus (NIDDM), neurodegenerative disorders, such as Alzheimer's Disease (AD), and the like. Thus, the present invention is directed to complexes of these proteins and/or their fragments, antibodies to the complexes, diagnosis of physiological generative disorders (including diagnosis of a predisposition to and diagnosis of the existence of the disorder), drug screening for agents which modulate the interaction of proteins described herein, and identification of additional proteins in the pathway common to the proteins described herein.

The publications and other materials used herein to illuminate the background of the invention, and in particular, cases to provide additional details respecting the practice, are incorporated herein by reference, and for convenience, are referenced by author and date in the following text and respectively grouped in the appended Bibliography.

Many processes in biology, including transcription, translation and metabolic or signal transduction pathways, are mediated by non-covalently associated protein complexes. The formation of protein-protein complexes or protein-DNA complexes produce the most efficient chemical machinery. Much of modem biological research is concerned with identifying proteins involved in cellular processes, determining their functions, and how, when and where they interact with other proteins involved in specific pathways. Further, with rapid advances in genome sequencing, there is a need to define protein linkage maps, i.e., detailed inventories of protein interactions that make up functional assemblies of proteins or protein complexes or that make up physiological pathways.

Recent advances in human genomics research has led to rapid progress in the identification of novel genes. In applications to biological and pharmaceutical research, there is a need to determine functions of gene products. A first step in defining the function of a novel gene is to determine its interactions with other gene products in appropriate context. That is, since proteins make specific interactions with other proteins or other biopolymers as part of functional assemblies or physiological pathways, an appropriate way to examine function of a gene is to determine its physical relationship with other genes. Several systems exist for identifying protein interactions and hence relationships between genes.

There continues to be a need in the art for the discovery of additional protein-protein interactions involved in mammalian physiological pathways. There continues to be a need in the art also to identify the protein-protein interactions that are involved in mammalian physiological disorders and diseases, and to thus identify drug targets.

### SUMMARY OF THE INVENTION

The present invention relates to the discovery of protein-protein interactions that are involved in mammalian physiological pathways, including physiological disorders or diseases, and to the use of this discovery. The identification of the interacting proteins described herein provide new targets for the identification of useful pharmaceuticals, new targets for diagnostic tools in the identification of individuals at risk, sequences for production of transformed cell lines, cellular models and animal models, and new bases for therapeutic intervention in such physiological pathways

Thus, one aspect of the present invention is protein complexes. The protein complexes are a complex of (a) two interacting proteins, (b) a first interacting protein and a fragment of a second interacting protein, (c) a fragment of a first interacting protein and a second interacting protein, or (d) a fragment of a first interacting protein and a fragment of a second interacting protein. The fragments of the interacting proteins include those parts of the proteins, which interact to form a complex. This aspect of the invention includes the detection of protein interactions and the production of proteins by recombinant techniques. The latter embodiment also includes cloned sequences, vectors, transfected or transformed host cells and transgenic animals.

A second aspect of the present invention is an antibody that is immunoreactive with the above complex. The antibody may be a polyclonal antibody or a monoclonal antibody. While the antibody is immunoreactive with the complex, it is not immunoreactive with the component parts of the complex. That is, the antibody is not immunoreactive with a first interactive protein, a fragment of a first interacting protein, a second interacting protein or a fragment of a second interacting protein. Such antibodies can be used to detect the presence or absence of the protein complexes.

A third aspect of the present invention is a method for diagnosing a predisposition for physiological disorders or diseases in a human or other animal. The diagnosis of such disorders includes a diagnosis of a predisposition to the disorders and a diagnosis for the existence of the disorders. In accordance with this method, the ability of a first interacting protein or fragment thereof to form a complex with a second interacting protein or a fragment thereof is assayed, or the genes encoding interacting proteins are screened for mutations in interacting portions of the protein molecules. The inability of a first interacting protein or fragment thereof to form a complex, or the presence of mutations in a gene within the interacting domain, is indicative of a predisposition to, or existence of a disorder. In accordance with one embodiment of the invention, the ability to form a complex is assayed in a two-hybrid assay. In a first aspect of this embodiment, the ability to form a complex is assayed by a yeast two-hybrid assay. In a second aspect, the ability to form a complex is assayed by a mammalian two-hybrid assay. In a second embodiment, the ability to form a complex is assayed by measuring in vitro a complex formed by combining said first protein and said second protein. In one aspect the proteins are isolated from a human or other animal. In a third embodiment, the ability to form a complex is assayed by measuring the binding of an antibody, which is specific for the complex. In a fourth embodiment, the ability to form a complex is assayed by measuring the binding of an antibody that is specific for the complex with a tissue extract from a human or other animal. In a fifth embodiment, coding sequences of the interacting proteins described herein are screened for mutations.

A fourth aspect of the present invention is a method for screening for drug candidates which are capable of modulating the interaction of a first interacting protein and a second interacting protein. In this method, the amount of the complex formed in the presence of a drug is compared with the amount of the complex formed in the absence of the drug. If the amount of complex formed in the presence of the drug is greater than or less than the amount of complex formed in the absence of the drug, the drug is a candidate for modulating the interaction of the first and second interacting proteins. The drug promotes the interaction if the complex formed in the presence of the drug is greater and inhibits (or disrupts) the interaction if the complex formed in the presence of the drug is less. The drug may affect the interaction directly, i.e., by modulating the binding of the two proteins, or indirectly, e.g., by modulating the expression of one or both of the proteins.

A fifth aspect of the present invention is a model for such physiological pathways, disorders or diseases. The model may be a cellular model or an animal model, as further described herein. In accordance with one embodiment of the invention, an animal model is prepared by creating transgenic or “knock-out” animals. The knock-out may be a total knock-out, i.e., the desired gene is deleted, or a conditional knock-out, i.e., the gene is active until it is knocked out at a determined time. In a second embodiment, a cell line is derived from such animals for use as a model. In a third embodiment, an animal model is prepared in which the biological activity of a protein complex of the present invention has been altered. In one aspect, the biological activity is altered by disrupting the formation of the protein complex, such as by the binding of an antibody or small molecule to one of the proteins which prevents the formation of the protein complex. In a second aspect, the biological activity of a protein complex is altered by disrupting the action of the complex, such as by the binding of an antibody or small molecule to the protein complex which interferes with the action of the protein complex as described herein. In a fourth embodiment, a cell model is prepared by altering the genome of the cells in a cell line. In one aspect, the genome of the cells is modified to produce at least one protein complex described herein. In a second aspect, the genome of the cells is modified to eliminate at least one protein of the protein complexes described herein.

A sixth aspect of the present invention are nucleic acids coding for novel proteins discovered in accordance with the present invention and the corresponding proteins and antibodies.

A seventh aspect of the present invention is a method of screening for drug candidates useful for treating a physiological disorder. In this embodiment, drugs are screened on the basis of the association of a protein with a particular physiological disorder. This association is established in accordance with the present invention by identifying a relationship of the protein with a particular physiological disorder. The drugs are screened by comparing the activity of the protein in the presence and absence of the drug. If a difference in activity is found, then the drug is a drug candidate for the physiological disorder. The activity of the protein can be assayed in vitro or in vivo using conventional techniques, including transgenic animals and cell lines of the present invention.

### DETAILED DESCRIPTION OF THE INVENTION

The present invention is the discovery of novel interactions between proteins described herein. The genes coding for some of these proteins may have been cloned previously, but their potential interaction in a physiological pathway or with a particular protein was unknown. Alternatively, the genes coding for some of these proteins have not been cloned previously and represent novel genes. These proteins are identified using the yeast two-hybrid method and searching a human total brain library, as more fully described below.

According to the present invention, new protein-protein interactions have been discovered. The discovery of these interactions has identified several protein complexes for each protein-protein interaction. The protein complexes for these interactions are set forth below in Tables 1-11, which also identify the new protein-protein interactions of the present invention.

TABLE 1
Protein Complexes of MAPKAP-K3/KIAA0796 Interaction

MAP Kinase MAPKAP-K3 (MAPKAP-K3) and KIAA0796
A fragment of MAPKAP-K3 and KIAA0796
MAPKAP-K3 and a fragment of KIAA0796
A fragment of MAPKAP-K3 and a fragment of KIAA0796

TABLE 2
Protein Complexes of MAPKAP-K3/USF2 Interaction

MAP Kinase MAPKAP-K3 (MAPKAP-K3) and Upstream Stimulatory
Factor 2 (USF2)
A fragment of MAPKAP-K3 and USF2
MAPKAP-K3 and a fragment of USF2
A fragment of MAPKAP-K3 and a fragment of USF2

TABLE 3
Protein Complexes of MAPKAP-K3/MLCK Interaction

MAP Kinase MAPKAP-K3 (MAPKAP-K3) and Myosin Light Chain
Kinase (MLCK)
A fragment of MAPKAP-K3 and MLCK
MAPKAP-K3 and a fragment of MLCK
A fragment of MAPKAP-K3 and a fragment of MLCK

TABLE 4
Protein Complexes of MAPKAP-K3/Hevin Interaction

MAP Kinase MAPKAP-K3 (MAPKAP-K3) and Hevin
A fragment of MAPKAP-K3 and Hevin
MAPKAP-K3 and a fragment of Hevin
A fragment of MAPKAP-K3 and a fragment of Hevin

TABLE 5
Protein Complexes of L130/Golgin-160 Interaction

Leucine Rich Protein L130 (L130) and Golgin-160
A fragment of L130 and Golgin-160
L130 and a fragment of Golgin-160
A fragment of L130 and a fragment of Golgin-160

TABLE 6
Protein Complexes of L130/Alpha-Spectin Interaction

Leucine Rich Protein L130 (L130) and Alpha-Spectin
A fragment of L130 and Alpha-Spectin
L130 and a fragment of Alpha-Spectin
A fragment of L130 and a fragment of Alpha-Spectin

TABLE 7
Protein Complexes of PRAK/Prox1 Interaction

Protein Kinase PRAK (PRAK) and Prox1
A fragment of PRAK and Prox1
PRAK and a fragment of Prox1
A fragment of PRAK and a fragment of Prox1

TABLE 8
Protein Complexes of PRAK/AL117237 Interaction

Protein Kinase PRAK (PRAK) and AL117237
A fragment of PRAK and AL117237
PRAK and a fragment of AL117237
A fragment of PRAK and a fragment of AL117237

TABLE 9
Protein Complexes of p38 alpha/14-3-3-Beta Interaction

Protein Kinase p38 alpha (p38 alpha) and 14-3-3-beta
A fragment of p38 alpha and 14-3-3-beta
p38 alpha and a fragment of 14-3-3-beta
A fragment of p38 alpha and a fragment of 14-3-3-beta

TABLE 10
Protein Complexes of ERK3/Restin Interaction

ERK3 and Restin/CLIP170 (Restin)
A fragment of ERK3 and Restin
ERK3 and a fragment of Restin
A fragment of ERK3 and a fragment of Restin

TABLE 11
Protein Complexes of ERK3/SEI1 Interaction

ERK3 and SE11
A fragment of ERK3 and SEI1
ERK3 and a fragment of SEI1
A fragment of ERK3 and a fragment of SEI1

The involvement of above interactions in particular pathways is as follows.

Many cellular proteins exert their function by interacting with other proteins in the cell. Examples of this are found in the formation of multiprotein complexes and the association of an enzymes with their substrates. It is widely believed that a great deal of information can be gained by understanding individual protein-protein interactions, and that this is useful in identifying complex networks of interacting proteins that participate in the workings of normal cellular functions. Ultimately, the knowledge gained by characterizing these networks can lead to valuable insight into the causes of human diseases and can eventually lead to the development of therapeutic strategies. The yeast two-hybrid assay is a powerful tool for determining protein-protein interactions and it has been successfully used for studying human disease pathways. In one variation of this technique, a protein of interest (or a portion of that protein) is expressed in a population of yeast cells that collectively contain all protein sequences. Yeast cells that possess protein sequences that interact with the protein of interest are then genetically selected, and the identity of those interacting proteins are determined by DNA sequencing. Thus, proteins that can be demonstrated to interact with a protein known to be involved in a human disease are therefore also implicated in that disease. To create a more complex network of interactions in a disease pathway, proteins which were identified in the first round of two-hybrid screening are subsequently used in two-hybrid assays as the protein of interest.

Cellular events that are initiated by exposure to growth factors, cytokines and stress are propagated from the outside of the cell to the nucleus by means of several protein kinase signal transduction cascades. p38 kinase is a member of the MAP kinase family of protein kinases. It is a key player in signal transduction pathways induced by the proinflammatory cytokines such as tumor necrosis factor (TNF), interleukin-1 (IL-1) and interleukin-6 (IL-6) and it also plays a critical role in the synthesis and release of the proinflammatory cytokines (Raingeaud et al., 1995; Lee et al., 1996; Miyazawa et al., 1998; Lee et al., 1994). Studies of inhibitors of p38 kinase have shown that blocking p38 kinase activity can cause anti-inflammatory effects, thus suggesting that this may be a mechanism of treating certain inflammatory diseases such as rheumatoid arthritis and inflammatory bowel disease. Further, p38 kinase activity has been implicated in other human diseases such as atherosclerosis, cardiac hypertrophy and hypoxic brain injury (Grammer et al., 1998; Mach et al., 1998; Wang et al., 1998; Nemoto et al., 1998; Kawasaki et al., 1997). Thus, by understanding p38 function, one may gain novel insight into a cellular response mechanism that affects a number of tissues and can potentially lead to harmful affects when disrupted.

The search for the physiological substrates of p38 kinase has taken a number of approaches including a variety of biochemical and cell biological methods. There are four known human isoforms of p38 kinase termed alpha, beta, gamma and delta, and these are thought to possess different physiological functions, likely because they have distinct substrate and tissue specificities. Some of the p38 kinase substrates are known, and the list includes transcription factors and additional protein kinases that act downstream of p38 kinase. Four of the kinases that act downstream of p38 kinase, MAPKAP-K2, MAPKAP-K3, PRAK and MSK1, are currently being analyzed themselves and some of their substrates have been identified.

The yeast two-hybrid system has been used to detect potential substrates and upstream regulators of the p38 kinases and their downstream kinases. In a two-hybrid search using p38 alpha kinase as the protein of interest, JNK1 was shown to bind to p38 alpha. JNK1 is itself a member of the MAP kinase family like the p38 kinases. JNK1 is induced by exposure to UV light, IL-1 and Ha-ras, and activation of JNK1 enzymatic activity requires phosphorylation of threonine and tyrosine residues at positions 183 and 185, respectively (Sluss et al., 1994). Inspection of the amino acid sequence of JNK1 reveals that there may be as many as 3 MAP kinase consensus phosphorylation sites, therefore it is quite possible that p38 alpha may utilize JNK1 as a substrate and may activate its kinase function. The finding that p38 alpha and JNK1 associate with one another is the third interaction of this kind to be found in the course of this project; we have also found that p38 alpha kinase interacts with the related MAP kinases JNK2 and JNK3 alpha2.

MAPKAP-K3, a protein kinase that acts downstream of p38 kinase in the same signal transduction pathway, was used in a two-hybrid search to identify potential substrates or regulators. MAPKAP-K3 was demonstrated to interact with four proteins in the yeast two-hybrid assay. The first protein, the transcription factor USF2 (upstream stimulatory factor 2), is a basic helix-loop-helip, leucine zipper-containing protein that is ubiquitously expressed. It has been suggested that USF2 may act as a negative regulator of cell proliferation since it has been shown that it can block E1A-mediated transformation and colony-formation in HeLa cells (Luo and Sawadago, 1996). USF2 has also been linked directly to cancer (Jaiswal and Narayan, 2001; Ismail et al., 1999), fertility (Heckert et al., 2000), angiogenesis (Harris et al., 2000), and to HIV (Moriuchi et la., 1999). Curiously, there do not appear to be any good MAPKAP consensus phosphorylation sites present in the USF2 protein sequence, so it is therefore difficult to draw any conclusions about the significance of the interaction between MAPKAP-K3 and USF2. Since USF2 is known to heterodimerize, it is possible that MAPKAP-K3 could bind to USF2 and phosphorylate its partner. Curiously, there do not appear to be any good MAPKAP consensus phosphorylation sites present in the USF2 protein sequence, so it is therefore difficult to draw any conclusions about the significance of the interaction between MAPKAP-K3 and USF2. Since USF2 is known to heterodimerize, it is possible that MAPKAP-K3 could bind to USF2 and phosphorylate its partner.

The second protein shown to interact with MAPKAP-K3 is MLCK or myosin light chain kinase. MLCK phosphorylates a specific serine residue in the N-terminus of myosin light chain as an early step of muscle contraction, however it must play additional roles since an isoform of it is also present in non-muscle cells. In a very recent report looking at the regulation of polymorphonuclear leukocyte phagocytosis by MLCK, it has been suggested that ERK2 activates MLCK, which in turn activates myosin to induce critical cytoskeletal changes (Mansfield et al., 2000). Our finding that MAPKAP-K3 interacts with MLCK provides evidence that perhaps MAPKAP-K3 can also activate MLCK and induce such cytoskeletal rearrangements. Indeed, MLCK contains 3 MAPKAP consensus phosphorylation sites. Interestingly, a new function for MLCK has recently been described: vasodilation (Watanabe et al., 2001). It has been suggested that in this role, the substrate for MLCK is not myosin light chain. Thus, MAPKAP-K3 may be involved in the regulation of MLCK and its role in vasodilation.

The third protein demonstrated to interact with MAPKAP-K3 in the yeast two-hybrid assay is the secreted acidic glycoprotein protein called hevin. The hevin cDNA was first cloned from high endothelial venules (HEV), structures in lymphoid tissues that support lymphocyte extravasion from the blood (Girard and Springer, 1995). The hevin protein sequence is similar to that of the anti-adhesive extracellular matrix protein called osteonectin (or SPARC), although hevin appears to modulate adhesion of endothelial cells to the basement membrane (Girard and Springer, 1996). In more recent studies, hevin has been shown to be down-regulated in a number of human cancers; further, over-expression of hevin has been demonstrated to inhibit the progression of cells from the G1 phase of the cell cycle to S phase or to lengthen G1 (Claeskens et al., 2000). These observations, taken together with the observation that the hevin gene lies in a chromosomal region frequently deleted in human tumors, have led researchers to postulate that hevin is in fact a tumor suppressor. As interesting as hevin appears to be, the biological connection between hevin and MAPKAP-K3 is a bit difficult to discern since the two proteins should reside in different locations. However hevin does appear to have a single MAPKAP consensus phosphorylation site, so it may be capable of being phosphorylated by MAPKAP-K3 or another related protein kinase.

Finally, in a two-hybrid search using MAPKAP-K3 as a protein of interest, a protein of unknown function called KIAA0796 was found to be an interactor. KIAA0796 is likely the human homolog of the mouse Syne-1b protein since it is 90% identical at the amino acid level. Syne-1b function is unknown but it is selectively associated with synaptic nuclei (Apel et al., 2000). Like Syne-1b, KIAA0796 contains a nuclear localization sequence and spectrin repeats. ESTs (expressed sequence tags) that are homologous to KIAA0796 have been found in a variety of tissues suggesting that it may be widely expressed. KIAA0796 may be capable of acting as a substrate for MAPKAP-K3 since there appears to be at least one MAPKAP consensus phosphorylation site in the known (partial) amino acid sequence.

When a second p38-activated protein kinase, PRAK, was used in a two-hybrid search two proteins were demonstrated to bind to it. The first protein, the Prox1 transcription factor, is a homeobox-containing protein similar to the Drosophila prospero protein. It is expressed in a number of embryonic and adult tissues. Prox1 has been well studied in mice, and it has been shown to be necessary for the development of the mouse lymphatic system (Wigle et al., 1999). PRAK may be capable of phosphorylating Prox1, thereby affecting its transcriptional function. Since the Prox1 amino acid sequence contains three putative MAPKAP consensus phosphorylation sites, it is likely that PRAK can indeed phosphorylate Prox1. The interaction between PRAK and Prox1 has been previously reported by us using the wild-type version of PRAK, however in this case, the interaction between the T182D mutant of PRAK was used. Interestingly, PRAK and Prox1 have been demonstrated by us to have a common interactor, the MLK2 mixed lineage kinase. MLK2 is involved in signal transduction like PRAK and it is a serine/threonine kinase with similarity to the MAP3K family of kinases. The finding that PRAK and Prox1 have a common interactor suggests that all three proteins may be involved in some common process and that perhaps they form a multiprotein complex.

The second protein found to interact with the T182D version of PRAK, and previously shown to interact with wild-type PRAK, is encoded by the AL117237 hypothetical protein sequence (formerly listed under GenBank accession number AL050141 and referred to as clone DKFZp586O031). The new GenBank reference is a composite of public ESTs and first pass sequences, and it predicts a protein of 921 amino acids. This predicted protein has no obvious structural domains other than 4 putative bipartite nuclear localization sequences. Interestingly, it does contain 7 MAPKAP consensus phosphorylation sites, suggesting strongly that it may be a substrate of one or more of the MAPKAP kinases such as PRAK. Based on the inspection of the ESTs that make up the composite sequence, the AL117237 protein product is predicted to be expressed in a very wide variety of tissues.

Yeast two-hybrid searches have been performed using a leucine-rich protein of unknown function called L130 that was previously identified by us to be a common interactor of both MAPKAP-K2 and PRAK. L130 was originally identified by virtue of its high level of expression in hepatoblastoma cells (Hou et al., 1994), however there is currently no information about its function. Its expression in hepatoblastoma cells suggests a role in liver function or in the transformation of normal cells to malignant ones. L130 has been shown to interact with two proteins in two-hybrid searches. The first protein, golgin-160, is a protein of the Golgi apparatus that is a human autoantigen (Fritzler et al., 1993). Its function is largely unknown but it is suspected to play a role in vesicular transport of proteins since it is localized to the Golgi appartus. Recent findings have shown that golgin-160 is cleaved by caspase-2 during apoptosis (Mancini et al., 2000).

The second protein shown to bind to L130 is the structural protein alpha-spectrin. Spectrins give flexibility to the cell and also act as a scaffold for other cellular proteins (Grum et al., 1999). Alpha-spectrin is a spectrin repeat-containing cytoskeletal protein that is thought to play a role in secretion. Alpha-spectrin has recently been tied to inflammation and apoptosis since it has beend demonstrated that TNF-alpha stimulates caspase-3 activation which in turn causes alpha-spectrin cleavage and apoptosis (Zhao et al., 2001). L130 has also been shown to associate with non-erythrocytic beta-spectrin. Interestingly, in our previous studies, we have shown that alpha-spectrin also binds to MAPKAP-K3 in the yeast two-hybrid assay.

In our previous findings, the ERK3 protein kinase was shown to interact with PRAK. ERK3 is a serine/threonine protein kinase of relatively unknown function (Cheng et al., 1996). It is a nuclear protein present in several tissues and is expressed in response to a number of extracellular stimuli. In two-hybrid searches using ERK3 as a protein of interest, two proteins were shown to be interactors. The first protein, SEI1, was first described as a regulator of the cyclin-dependent kinase CDK4 (Sugimoto et al., 1999). SEI1 is rapidly induced in serum-stimulated cells, and it appears to function by antagonizing the activity of the CDK inhibitor p16(INK4a). The finding that SEI1 and ERK3 interact suggests that ERK3 may be capable of phosphorylating SEI1 and thereby affecting its function. Alternatively, SEI1 may regulate the function of ERK3 in the same manner as it affects CDK4 function. Interestingly, SEI1 was previously shown by us to interact with the TIAR protein which has been implicated in the regulation TNF (tumor necrosis factor) alpha message (Gueydan et al., 1999).

ERK3 has also been shown to interact with the restin/CLIP 170 protein in the yeast two-hybrid assay. Restin is a cytoplasmic protein that is thought to link endocytic vesicles with microtubules (Pierre et al., 1992). A recent report indicates that restin colocalizes at microtubule plus ends with dynactin, a protein that we have shown to interact with PRAK and L130 (Valetti et al., 1999). To make things more complicated, PRAK itself interacts with L130 and ERK3. Taken together, it is tempting to speculate that some or all of these proteins may be capable of forming a larger, multiprotein complex that functions in vesicular transport. Perhaps the two associated kinases, ERK3 and PRAK, serve to regulate the function of such a large complex. As an interesting side note, restin is highly expressed in Reed-Stemberg cells of Hodgkin's disease, suggesting that it may play a key role in lymphocyte function (Bilbe et al., 1992).

The proteins disclosed in the present invention were found to interact with their corresponding proteins in the yeast two-hybrid system. Because of the involvement of the corresponding proteins in the physiological pathways disclosed herein, the proteins disclosed herein also participate in the same physiological pathways. Therefore, the present invention provides a list of uses of these proteins and DNA encoding these proteins for the development of diagnostic and therapeutic tools useful in the physiological pathways. This list includes, but is not limited to, the following examples.

Two-Hybrid System

The principles and methods of the yeast two-hybrid system have been described in detail elsewhere (e.g., Bartel and Fields, 1997; Bartel et al., 1993; Fields and Song, 1989; Chevray and Nathans, 1992). The following is a description of the use of this system to identify proteins that interact with a protein of interest.

The target protein is expressed in yeast as a fusion to the DNA-binding domain of the yeast Gal4p. DNA encoding the target protein or a fragment of this protein is amplified from cDNA by PCR or prepared from an available clone. The resulting DNA fragment is cloned by ligation or recombination into a DNA-binding domain vector (e.g., pGBT9, pGBT.C, pAS2-1) such that an in-frame fusion between the Gal4p and target protein sequences is created.

The target gene construct is introduced, by transformation, into a haploid yeast strain. A library of activation domain fusions (i.e., adult brain cDNA cloned into an activation domain vector) is introduced by transformation into a haploid yeast strain of the opposite mating type. The yeast strain that carries the activation domain constructs contains one or more Gal4p-responsive reporter gene(s), whose expression can be monitored. Examples of some yeast reporter strains include Y190, PJ69, and CBY14a. An aliquot of yeast carrying the target gene construct is combined with an aliquot of yeast carrying the activation domain library. The two yeast strains mate to form diploid yeast and are plated on media that selects for expression of one or more Gal4p-responsive reporter genes. Colonies that arise after incubation are selected for further characterization.

The activation domain plasmid is isolated from each colony obtained in the two-hybrid search. The sequence of the insert in this construct is obtained by the dideoxy nucleotide chain termination method. Sequence information is used to identify the gene/protein encoded by the activation domain insert via analysis of the public nucleotide and protein databases. Interaction of the activation domain fusion with the target protein is confirmed by testing for the specificity of the interaction. The activation domain construct is co-transformed into a yeast reporter strain with either the original target protein construct or a variety of other DNA-binding domain constructs. Expression of the reporter genes in the presence of the target protein but not with other test proteins indicates that the interaction is genuine.

In addition to the yeast two-hybrid system, other genetic methodologies are available for the discovery or detection of protein-protein interactions. For example, a mammalian two-hybrid system is available commercially (Clontech, Inc.) that operates on the same principle as the yeast two-hybrid system. Instead of transforming a yeast reporter strain, plasmids encoding DNA-binding and activation domain fusions are transfected along with an appropriate reporter gene (e.g., 1acZ) into a mammalian tissue culture cell line. Because transcription factors such as the Saccharomyces cerevisiae Gal4p are functional in a variety of different eukaryotic cell types, it would be expected that a two-hybrid assay could be performed in virtually any cell line of eukaryotic origin (e.g., insect cells (SF9), fungal cells, worm cells, etc.). Other genetic systems for the detection of protein-protein interactions include the so-called SOS recruitment system (Aronheim et al., 1997).

Protein-protein Interactions

Protein interactions are detected in various systems including the yeast two-hybrid system, affinity chromatography, co-immunoprecipitation, subcellular fractionation and isolation of large molecular complexes. Each of these methods is well characterized and can be readily performed by one skilled in the art. See, e.g., U.S. Pat. Nos. 5,622,852 and 5,773,218, and PCT published application No. WO 97/27296 and PCT published application No. WO 99/65939, each of which are incorporated herein by reference.

The protein of interest can be produced in eukaryotic or prokaryotic systems. A cDNA encoding the desired protein is introduced in an appropriate expression vector and transfected in a host cell (which could be bacteria, yeast cells, insect cells, or mammalian cells). Purification of the expressed protein is achieved by conventional biochemical and immunochemical methods well known to those skilled in the art. The purified protein is then used for affinity chromatography studies: it is immobilized on a matrix and loaded on a column. Extracts from cultured cells or homogenized tissue samples are then loaded on the column in appropriate buffer, and non-binding proteins are eluted. After extensive washing, binding proteins or protein complexes are eluted using various methods such as a gradient of pH or a gradient of salt concentration. Eluted proteins can then be separated by two-dimensional gel electrophoresis, eluted from the gel, and identified by micro-sequencing. The purified proteins can also be used for affinity chromatography to purify interacting proteins disclosed herein. All of these methods are well known to those skilled in the art.

Similarly, both proteins of the complex of interest (or interacting domains thereof) can be produced in eukaryotic or prokaryotic systems. The proteins (or interacting domains) can be under control of separate promoters or can be produced as a fusion protein. The fusion protein may include a peptide linker between the proteins (or interacting domains) which, in one embodiment, serves to promote the interaction of the proteins (or interacting domains). All of these methods are also well known to those skilled in the art.

Purified proteins of interest, individually or a complex, can also be used to generate antibodies in rabbit, mouse, rat, chicken, goat, sheep, pig, guinea pig, bovine, and horse. The methods used for antibody generation and characterization are well known to those skilled in the art. Monoclonal antibodies are also generated by conventional techniques. Single chain antibodies are further produced by conventional techniques.

DNA molecules encoding proteins of interest can be inserted in the appropriate expression vector and used for transfection of eukaryotic cells such as bacteria, yeast, insect cells, or mammalian cells, following methods well known to those skilled in the art. Transfected cells expressing both proteins of interest are then lysed in appropriate conditions, one of the two proteins is immunoprecipitated using a specific antibody, and analyzed by polyacrylamide gel electrophoresis. The presence of the binding protein (co-immunoprecipitated) is detected by immunoblotting using an antibody directed against the other protein. Co-immunoprecipitation is a method well known to those skilled in the art.

Transfected eukaryotic cells or biological tissue samples can be homogenized and fractionated in appropriate conditions that will separate the different cellular components. Typically, cell lysates are run on sucrose gradients, or other materials that will separate cellular components based on size and density. Subcellular fractions are analyzed for the presence of proteins of interest with appropriate antibodies, using immunoblotting or immunoprecipitation methods. These methods are all well known to those skilled in the art.

Disruption of Protein-protein Interactions

It is conceivable that agents that disrupt protein-protein interactions can be beneficial in many physiological disorders, including, but not-limited to NIDDM, AD and others disclosed herein. Each of the methods described above for the detection of a positive protein-protein interaction can also be used to identify drugs that will disrupt said interaction. As an example, cells transfected with DNAs coding for proteins of interest can be treated with various drugs, and co-immunoprecipitations can be performed. Alternatively, a derivative of the yeast two-hybrid system, called the reverse yeast two-hybrid system (Leanna and Hannink, 1996), can be used, provided that the two proteins interact in the straight yeast two-hybrid system.

Modulation of Protein-protein Interactions

Since the interaction described herein is involved in a physiological pathway, the identification of agents which are capable of modulating the interaction will provide agents which can be used to track the physiological disorder or to use as lead compounds for development of therapeutic agents. An agent may modulate expression of the genes of interacting proteins, thus affecting interaction of the proteins. Alternatively, the agent may modulate the interaction of the proteins. The agent may modulate the interaction of wild-type with wild-type proteins, wild-type with mutant proteins, or mutant with mutant proteins. Agents can be tested using transfected host cells, cell lines, cell models or animals, such as described herein, by techniques well known to those of ordinary skill in the art, such as disclosed in U.S. Pat. Nos. 5,622,852 and 5,773,218, and PCT published application No. WO 97/27296 and PCT published application No. WO 99/65939, each of which are incorporated herein by reference. The modulating effect of the agent can be screened in vivo or in vitro. Exemplary of a method to screen agents is to measure the effect that the agent has on the formation of the protein complex.

Mutation Screening

The proteins disclosed in the present invention interact with one or more proteins known to be involved in a physiological pathway, such as in NIDDM or AD. Mutations in interacting proteins could also be involved in the development of the physiological disorder, such as NIDDM or AD, for example, through a modification of protein-protein interaction, or a modification of enzymatic activity, modification of receptor activity, or through an unknown mechanism. Therefore, mutations can be found by sequencing the genes for the proteins of interest in patients having the physiological disorder, such as insulin, and non-affected controls. A mutation in these genes, especially in that portion of the gene involved in protein interactions in the physiological pathway, can be used as a diagnostic tool and the mechanistic understanding the mutation provides can help develop a therapeutic tool.

Screening for At-risk Individuals

Individuals can be screened to identify those at risk by screening for mutations in the protein disclosed herein and identified as described above. Alternatively, individuals can be screened by analyzing the ability of the proteins of said individual disclosed herein to form natural complexes. Further, individuals can be screened by analyzing the levels of the complexes or individual proteins of the complexes or the mRNA encoding the protein members of the complexes. Techniques to detect the formation of complexes, including those described above, are known to those skilled in the art. Techniques and methods to detect mutations are well known to those skilled in the art. Techniques to detect the level of the complexes, proteins or MRNA are well known to those skilled in the art.

Cellular Models of Physiological Disorders

A number of cellular models of many physiological disorders or diseases have been generated. The presence and the use of these models are familiar to those skilled in the art. As an example, primary cell cultures or established cell lines can be transfected with expression vectors encoding the proteins of interest, either wild-type proteins or mutant proteins. The effect of the proteins disclosed herein on parameters relevant to their particular physiological disorder or disease can be readily measured. Furthermore, these cellular systems can be used to screen drugs that will influence those parameters, and thus be potential therapeutic tools for the particular physiological disorder or disease. Alternatively, instead of transfecting the DNA encoding the protein of interest, the purified protein of interest can be added to the culture medium of the cells under examination, and the relevant parameters measured.

Animal Models

The DNA encoding the protein of interest can be used to create animals that overexpress said protein, with wild-type or mutant sequences (such animals are referred to as “transgenic”), or animals which do not express the native gene but express the gene of a second animal (referred to as “transplacement”), or animals that do not express said protein (referred to as “knock-out”). The knock-out animal may be an animal in which the gene is knocked out at a determined time. The generation of transgenic, transplacement and knock-out animals (normal and conditioned) uses methods well known to those skilled in the art.

In these animals, parameters relevant to the particular physiological disorder can be measured. These parametes may include receptor function, protein secretion in vivo or in vitro, survival rate of cultured cells, concentration of particular protein in tissue homogenates, signal transduction, behavioral analysis, protein synthesis, cell cycle regulation, transport of compounds across cell or nuclear membranes, enzyme activity, oxidative stress, production of pathological products, and the like. The measurements of biochemical and pathological parameters, and of behavioral parameters, where appropriate, are performed using methods well known to those skilled in the art. These transgenic, transplacement and knock-out animals can also be used to screen drugs that may influence the biochemical, pathological, and behavioral parameters relevant to the particular physiological disorder being studied. Cell lines can also be derived from these animals for use as cellular models of the physiological disorder, or in drug screening.

Rational Drug Design

The goal of rational drug design is to produce structural analogs of biologically active polypeptides of interest or of small molecules with which they interact (e.g., agonists, antagonists, inhibitors) in order to fashion drugs which are, for example, more active or stable forms of the polypeptide, or which, e.g., enhance or interfere with the function of a polypeptide in vivo. Several approaches for use in rational drug design include analysis of three-dimensional structure, alanine scans, molecular modeling and use of anti-id antibodies. These techniques are well known to those skilled in the art.

Following identification of a substance which modulates or affects polypeptide activity, the substance may be further investigated. Furthermore, it may be manufactured and/or used in preparation, i.e., manufacture or formulation, or a composition such as a medicament, pharmaceutical composition or drug. These may be administered to individuals.

A substance identified as a modulator of polypeptide function may be peptide or non-peptide in nature. Non-peptide “small molecules” are often preferred for many in vivo pharmaceutical uses. Accordingly, a mimetic or mimic of the substance (particularly if a peptide) may be designed for pharmaceutical use.

The designing of mimetics to a known pharmaceutically active compound is a known approach to the development of pharmaceuticals based on a “lead” compound. This approach might be desirable where the active compound is difficult or expensive to synthesize or where it is unsuitable for a particular method of administration, e.g., pure peptides are unsuitable active agents for oral compositions as they tend to be quickly degraded by proteases in the alimentary canal. Mimetic design, synthesis and testing are generally used to avoid randomly screening large numbers of molecules for a target property.

Once the pharmacophore has been found, its structure is modeled according to its physical properties, e.g., stereochemistry, bonding, size and/or charge, using data from a range of sources, e.g., spectroscopic techniques, x-ray diffraction data and NMR. Computational analysis, similarity mapping (which models the charge and/or volume of a pharmacophore, rather than the bonding between atoms) and other techniques can be used in this modeling process.

A template molecule is then selected, onto which chemical groups that mimic the pharmacophore can be grafted. The template molecule and the chemical groups grafted thereon can be conveniently selected so that the mimetic is easy to synthesize, is likely to be pharmacologically acceptable, and does not degrade in vivo, while retaining the biological activity of the lead compound. Alternatively, where the mimetic is peptide-based, further stability can be achieved by cyclizing the peptide, increasing its rigidity. The mimetic or mimetics found by this approach can then be screened to see whether they have the target property, or to what extent it is exhibited. Further optimization or modification can then be carried out to arrive at one or more final mimetics for in vivo or clinical testing.

Diagnostic Assays

The identification of the interactions disclosed herein enables the development of diagnostic assays and kits, which can be used to determine a predisposition to or the existence of a physiological disorder. In one aspect, one of the proteins of the interaction is used to detect the presence of a “normal” second protein (i.e., normal with respect to its ability to interact with the first protein) in a cell extract or a biological fluid, and further, if desired, to detect the quantitative level of the second protein in the extract or biological fluid. The absence of the “normal” second protein would be indicative of a predisposition or existence of the physiological disorder. In a second aspect, an antibody against the protein complex is used to detect the presence and/or quantitative level of the protein complex. The absence of the protein complex would be indicative of a predisposition or existence of the physiological disorder.

Nucleic Acids and Proteins

A nucleic acid or fragment thereof has substantial identity with another if, when optimally aligned (with appropriate nucleotide insertions or deletions) with the other nucleic acid (or its complementary strand), there is nucleotide sequence identity in at least about 60% of the nucleotide bases, usually at least about 70%, more usually at least about 80%, preferably at least about 90%, and more preferably at least about 95-98% of the nucleotide bases. A protein or fragment thereof has substantial identity with another if, optimally aligned, there is an amino acid sequence identity of at least about 30% identity with an entire naturally-occurring protein or a portion thereof, usually at least about 70% identity, more usually at least about 80% identity, preferably at least about 90% identity, and more preferably at least about 95% identity.

Identity means the degree of sequence relatedness between two polypeptide or two polynucleotides sequences as determined by the identity of the match between two strings of such sequences, such as the full and complete sequence. Identity can be readily calculated. While there exist a number of methods to measure identity between two polynucleotide or polypeptide sequences, the term “identity” is well known to skilled artisans (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991). Methods commonly employed to determine identity between two sequences include, but are not limited to those disclosed in Guide to Huge Computers, Martin J. Bishop, ed., Academic Press, San Diego, 1994, and Carillo, H., and Lipman, D., SIAM J Applied Math. 48: 1073 (1988). Preferred methods to determine identity are designed to give the largest match between the two sequences tested. Such methods are codified in computer programs. Preferred computer program methods to determine identity between two sequences include, but are not limited to, GCG (Genetics Computer Group, Madison Wis.) program package (Devereux, J., et al., Nucleic Acids Research 12(1). 387 (1984)), BLASTP, BLASTN, FASTA (Altschul et al. (1990); Altschul et al. (1997)). The well-known Smith Waterman algorithm may also be used to determine identity.

As an illustration, by a polynucleotide having a nucleotide sequence having at least, for example, 95% “identity” to a reference nucleotide sequence of is intended that the nucleotide sequence of the polynucleotide is identical to the reference sequence except that the polynucleotide sequence may include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These mutations of the reference sequence may occur at the 5 or 3 terminal positions of the reference nucleotide sequence or anywhere between those terminal positions, interspersed either individually among nucleotides in the reference sequence or in one or more contiguous groups within the reference sequence.

Alternatively, substantial homology or (similarity) exists when a nucleic acid or fragment thereof will hybridize to another nucleic acid (or a complementary strand thereof) under selective hybridization conditions, to a strand, or to its complement. Selectivity of hybridization exists when hybridization which is substantially more selective than total lack of specificity occurs. Typically, selective hybridization will occur when there is at least about 55% homology over a stretch of at least about 14 nucleotides, preferably at least about 65%, more preferably at least about 75%, and most preferably at least about 90%. The length of homology comparison, as described, may be over longer stretches, and in certain embodiments will often be over a stretch of at least about nine nucleotides, usually at least about 20 nucleotides, more usually at least about 24 nucleotides, typically at least about 28 nucleotides, more typically at least about 32 nucleotides, and preferably at least about 36 or more nucleotides.

Nucleic acid hybridization will be affected by such conditions as salt concentration, temperature, or organic solvents, in addition to the base composition, length of the complementary strands, and the number of nucleotide base mismatches between the hybridizing nucleic acids, as will be readily appreciated by those skilled in the art. Stringent temperature conditions will generally include temperatures in excess of 30° C., typically in excess of 37° C., and preferably in excess of 45° C. Stringent salt conditions will ordinarily be less than 1000 mM, typically less than 500 mM, and preferably less than 200 mM. However, the combination of parameters is much more important than the measure of any single parameter. The stringency conditions are dependent on the length of the nucleic acid and the base composition of the nucleic acid, and can be determined by techniques well known in the art. See, e.g., Asubel, 1992; Wetmur and Davidson, 1968.

Thus, as herein used, the term “stringent conditions” means hybridization will occur only if there is at least 95% and preferably at least 97% identity between the sequences. Such hybridization techniques are well known to those of skill in the art. Stringent hybridization conditions are as defined above or, alternatively, conditions under overnight incubation at 42° C. in a solution comprising: 50% formamide, 5×SSC (150 mM NaCl, 15 mM trisodium citrate), 50 mM sodium phosphate (pH7.6), 5×Denhardt's solution, 10% dextran sulfate, and 20 μg/ml denatured, sheared salmon sperm DNA, followed by washing the filters in 0.1×SSC at about 65° C.

The terms “isolated”, “substantially pure”, and “substantially homogeneous” are used interchangeably to describe a protein or polypeptide which has been separated from components which accompany it in its natural state. A monomeric protein is substantially pure when at least about 60 to 75% of a sample exhibits a single polypeptide sequence. A substantially pure protein will typically comprise about 60 to 90% W/W of a protein sample, more usually about 95%, and preferably will be over about 99% pure. Protein purity or homogeneity may be indicated by a number of means well known in the art, such as polyacrylamide gel electrophoresis of a protein sample, followed by visualizing a single polypeptide band upon staining the gel. For certain purposes, higher resolution may be provided by using HPLC or other means well known in the art which are utilized for purification.

Large amounts of the nucleic acids of the present invention may be produced by (a) replication in a suitable host or transgenic animals or (b) chemical synthesis using techniques well known in the art. Constructs prepared for introduction into a prokaryotic or eukaryotic host may comprise a replication system recognized by the host, including the intended polynucleotide fragment encoding the desired polypeptide, and will preferably also include transcription and translational initiation regulatory sequences operably linked to the polypeptide encoding segment. Expression vectors may include, for example, an origin of replication or autonomously replicating sequence (ARS) and expression control sequences, a promoter, an enhancer and necessary processing information sites, such as ribosome-binding sites, RNA splice sites, polyadenylation sites, transcriptional terminator sequences, and mRNA stabilizing sequences. Secretion signals may also be included where appropriate which allow the protein to cross and/or lodge in cell membranes, and thus attain its functional topology, or be secreted from the cell. Such vectors may be prepared by means of standard recombinant techniques well known in the art.

### EXAMPLES

The present invention is further detailed in the following Examples, which are offered by way of illustration and are not intended to limit the invention in any manner. Standard techniques well known in the art or the techniques specifically described below are utilized.

### Example 1

Yeast Two-hybrid System

The principles and methods of the yeast two-hybrid systems have been described in detail (Bartel and Fields, 1997). The following is thus a description of the particular procedure that we used, which was applied to all proteins.

The cDNA encoding the bait protein was generated by PCR from brain cDNA. Gene-specific primers were synthesized with appropriate tails added at their 5′ ends to allow recombination into the vector pGBTQ. The tail for the forward primer was 5′-GCAGGAAACAGCTATGACCATACAGTCAGCGGCCGCCACC-3′ (SEQ ID NO: 1) and the tail for the reverse primer was 5′-ACGGCCAGTCGCGTGGAGTGTTATGTCATGCGGCCGCTA-3′ (SEQ ID NO:2). The tailed PCR product was then introduced by recombination into the yeast expression vector pGBTQ, which is a close derivative of pGBTC (Bartel et al., 1996) in which the polylinker site has been modified to include M13 sequencing sites. The new construct was selected directly in the yeast J693 for its ability to drive tryptophane synthesis (genotype of this strain: Mat α, ade2, his3, leu2, trp1, URA3::GAL1-1acZ LYS2::GAL1-HIS3 gal4del gal80del cyhR2). In these yeast cells, the bait is produced as a C-terminal fusion protein with the DNA binding domain of the transcription factor Gal4 (amino acids 1 to 147). A total human brain (37 year-old male Caucasian) cDNA library cloned into the yeast expression vector pACT2 was purchased from Clontech (human brain MATCHMAKER cDNA, cat. # HL4004AH), transformed into the yeast strain J692 (genotype of this strain: Mat a, ade2, his3, leu2, trp1, URA3::GAL1-lacZ LYS2::GAL1-HIS3 gal4del gal80cyhR2), and selected for the ability to drive leucine synthesis. In these yeast cells, each cDNA is expressed as a fusion protein with the transcription activation domain of the transcription factor Gal4 (amino acids 768 to 881) and a 9 amino acid hemagglutinin epitope tag. J693 cells (Mat α type) expressing the bait were then mated with J692 cells ( Mat a type) expressing proteins from the brain library. The resulting diploid yeast cells expressing proteins interacting with the bait protein were selected for the ability to synthesize tryptophan, leucine, histidine, and β-galactosidase. DNA was prepared from each clone, transformed by electroporation into E. coli strain KC8 (Clontech KC8 electrocompetent cells, cat. # C2023-1), and the cells were selected on ampicillin-containing plates in the absence of either tryptophane (selection for the bait plasmid) or leucine (selection for the brain library plasmid). DNA for both plasmids was prepared and sequenced by di-deoxynucleotide chain termination method. The identity of the bait cDNA insert was confirmed and the cDNA insert from the brain library plasmid was identified using BLAST program against public nucleotides and protein databases. Plasmids from the brain library (preys) were then individually transformed into yeast cells together with a plasmid driving the synthesis of lamin fused to the Gal4 DNA binding domain. Clones that gave a positive signal after β-galactosidase assay were considered false-positives and discarded. Plasmids for the remaining clones were transformed into yeast cells together with plasmid for the original bait. Clones that gave a positive signal after β-galactosidase assay were considered true positives.

### Example 2

Identification of MAPKAP-K3/KIAA0796 Interaction

A yeast two-hybrid system as described in Example 1 using amino acids encoded by nucleotides 433-1003 of MAPKAP-K3 (GenBank (GB) accession no. U09578) as bait was performed. One clone that was identified by this procedure included amino acids encoded by nucleotides 1128-1467 of KIAA0796 (GB accession no. AB018339).

### Example 3

Identification of MAPKAP-K3/USF2 Interaction

A yeast two-hybrid system as described in Example 1 using amino acids encoded by nucleotides 433-1003 of MAPKAP-K3 (GB accession no. U09578) as bait was performed. One clone that was identified by this procedure included amino acids 183-288 of USF2 (SP accession no. 000671).

### Example 4

Identification of MAPKAP-K3/MLCK Interaction

A yeast two-hybrid system as described in Example 1 using amino acids encoded by nucleotides 433-1003 of MAPKAP-K3 (GB accession no. U09578) as bait was performed. One clone that was identified by this procedure included amino acids 291-419 of MLCK (SP accession no. Q14844).

### Example 5

Identification of MAPKAP-K3/MLCK Interaction

A yeast two-hybrid system as described in Example 1 using amino acids encoded by nucleotides 433-1003 of MAPKAP-K3 (GB accession no. U09578) as bait was performed. One clone that was identified by this procedure included amino acids encoded by nucleotides 1024-1624 of Hevin (GB accession no. X82157).

### Example 6

Identification of L130/Golgin-160 Interaction

A yeast two-hybrid system as described in Example 1 using amino acids 400-700 of L130 (SP accession no. P42704) as bait was performed. One clone that was identified by this procedure included amino acids 53-164 of Golgin 160 (SP accession no. Q08378).

### Example 7

Identification of L130/Alpha-Spectrin Interaction

A yeast two-hybrid system as described in Example 1 using amino acids 400-700 of L130 (SP accession no. P42704) as bait was performed. One clone that was identified by this procedure included amino acids 1492-1854 of Alpha-Spectrin (SP accession no. Q13813).

### Example 8

Identification of L130/Alpha-Spectrin Interaction

A yeast two-hybrid system as described in Example 1 using amino acids 800-1100 of L130 (SP accession no. P42704) as bait was performed. One clone that was identified by this procedure included amino acids 1492-1854 of Alpha-Spectrin (SP accession no. Q13813).

### Example 9

Identification of PRAK/Prox1 Interaction

A yeast two-hybrid system as described in Example 1 using amino acids encoded by nucleotides 201-1104 of PRAK (GB accession no. AF032437) with the T182D mutation as bait was performed. One clone that was identified by this procedure included amino acids 204-464 of Prox1 (SP accession no. Q92786).

### Example 10

Identification of PRAK/AL117237 Interaction

A yeast two-hybrid system as described in Example 1 using amino acids encoded by nucleotides 201-1104 of PRAK (GB accession no. AF032437) with the T182D mutation as bait was performed. One clone that was identified by this procedure included amino acids encoded by nucleotides 1220-1481 of AL117237 (GB accession no. AL117237).

### Example 11

Identification of p38 alpha/JNK1 Beta Interaction

A yeast two-hybrid system as described in Example 1 using amino acids 1-130 of p38 alpha (SP accession no. Q13083) as bait was performed. One clone that was identified by this procedure included amino acids 219-374 of JNK1 (SP accession no. P45983).

### Example 12

Identification of ERK3/Restin Interaction

A yeast two-hybrid system as described in Example 1 using amino acids 1-316 of ERK3 (SP accession no. Q16659) as bait was performed. One clone that was identified by this procedure included amino acids 842-1152 of Restin/Clip170 (SP accession no. P30622).

### Example 13

Identification of ERK3/SEI1 Interaction

A yeast two-hybrid system as described in Example 1 using amino acids 1-316 of ERK3 (SP accession no. Q16659) as bait was performed. One clone that was identified by this procedure included amino acids encoded by nucleotides 131-548 of SEI1 (GB accession no. AF117959).

### Example 14

Generation of Polyclonal Antibody Against Protein Complexes

As shown above, MAPKAP-K3 interacts with KIAA0796 to form a complex. A complex of the two proteins is prepared, e.g., by mixing purified preparations of each of the two proteins. If desired, the protein complex can be stabilized by cross-linking the proteins in the complex, by methods known to those of skill in the art. The protein complex is used to immunize rabbits and mice using a procedure similar to that described by Harlow et al. (1988). This procedure has been shown to generate Abs against various other proteins (for example, see Kraemer et al., 1993).

Briefly, purified protein complex is used as immunogen in rabbits. Rabbits are immunized with 100 μg of the protein in complete Freund's adjuvant and boosted twice in three-week intervals, first with 100 μg of immunogen in incomplete Freund's adjuvant, and followed by 100 μg of immunogen in PBS. Antibody-containing serum is collected two weeks thereafter. The antisera is preadsorbed with MAPKAP-K3 and KIAA0796, such that the remaining antisera comprises antibodies which bind conformational epitopes, i.e., complex-specific epitopes, present on the MAPKAP-K3-KIAA0796 complex but not on the monomers.

Polyclonal antibodies against each of the complexes set forth in Tables 2-11 are prepared in a similar manner by mixing the specified proteins together, immunizing an animal and isolating antibodies specific for the protein complex, but not for the individual proteins.

### Example 15

Generation of Monoclonal Antibodies Specific for Protein Complexes

Monoclonal antibodies are generated according to the following protocol. Mice are immunized with immunogen comprising MAPKAP-K3/KIAA0796 complexes conjugated to keyhole limpet hemocyanin using glutaraldehyde or EDC as is well known in the art. The complexes can be prepared as described in Example 14, and may also be stabilized by cross-linking. The immunogen is mixed with an adjuvant. Each mouse receives four injections of 10 to 100 μg of immunogen, and after the fourth injection blood samples are taken from the mice to determine if the serum contains antibody to the immunogen. Serum titer is determined by ELISA or RIA. Mice with sera indicating the presence of antibody to the immunogen are selected for hybridoma production.

Spleens are removed from immune mice and a single-cell suspension is prepared (Harlow et al., 1988). Cell fusions are performed essentially as described by Kohler et al. (1975). Briefly, P3.65.3 myeloma cells (American Type Culture Collection, Rockville, MD) or NS-1 myeloma cells are fused with immune spleen cells using polyethylene glycol as described by Harlow et al. (1988). Cells are plated at a density of 2×10⁵ cells/well in 96-well tissue culture plates. Individual wells are examined for growth, and the supernatants of wells with growth are tested for the presence of MAPKAP-K3/KIAA0796 complex-specific antibodies by ELISA or RIA using MAPKAP-K3/KIAA0796 complex as target protein. Cells in positive wells are expanded and subcloned to establish and confirm monoclonality.

Clones with the desired specificities are expanded and grown as ascites in mice or in a hollow fiber system to produce sufficient quantities of antibodies for characterization and assay development. Antibodies are tested for binding to MAPKAP-K3 alone or to KIAA0796 alone, to determine which are specific for the MAPKAP-K3/KIAA0796 complex as opposed to those that bind to the individual proteins.

Monoclonal antibodies against each of the complexes set forth in Tables 2-11 are prepared in a similar manner by mixing the specified proteins together, immunizing an animal, fusing spleen cells with myeloma cells and isolating clones which produce antibodies specific for the protein complex, but not for the individual proteins.

### Example 16

In vitro Identification of Modulators for Protein-protein Interactions

The present invention is useful in screening for agents that modulate the interaction of MAPKAP-K3 and KIAA0796. The knowledge that MAPKAP-K3 and KIAA0796 form a complex is useful in designing such assays. Candidate agents are screened by mixing MAPKAP-K3 and KIAA0796 (a) in the presence of a candidate agent, and (b) in the absence of the candidate agent. The amount of complex formed is measured for each sample. An agent modulates the interaction of MAPKAP-K3 and KIAA0796 if the amount of complex formed in the presence of the agent is greater than (promoting the interaction), or less than (inhibiting the interaction) the amount of complex formed in the absence of the agent. The amount of complex is measured by a binding assay, which shows the formation of the complex, or by using antibodies immunoreactive to the complex.

Briefly, a binding assay is performed in which immobilized MAPKAP-K3 is used to bind labeled KIAA0796. The labeled KIAA0796 is contacted with the immobilized MAPKAP-K3 under aqueous conditions that permit specific binding of the two proteins to form an MAPKAP-K3/KIAA0796 complex in the absence of an added test agent. Particular aqueous conditions may be selected according to conventional methods. Any reaction condition can be used as long as specific binding of MAPKAP-K3/KIAA0796 occurs in the control reaction. A parallel binding assay is performed in which the test agent is added to the reaction mixture. The amount of labeled KIAA0796 bound to the immobilized MAPKAP-K3 is determined for the reactions in the absence or presence of the test agent. If the amount of bound, labeled KIAA0796 in the presence of the test agent is different than the amount of bound labeled KIAA0796 in the absence of the test agent, the test agent is a modulator of the interaction of MAPKAP-K3 and KIAA0796.

Candidate agents for modulating the interaction of each of the protein complexes set forth in Tables 2-11 are screened in vitro in a similar manner.

### Example 17

In vivo Identification of Modulators for Protein-protein Interactions

In addition to the in vitro method described in Example 16, an in vivo assay can also be used to screen for agents which modulate the interaction of MAPKAP-K3 and KIAA0796. Briefly, a yeast two-hybrid system is used in which the yeast cells express (1) a first fusion protein comprising MAPKAP-K3 or a fragment thereof and a first transcriptional regulatory protein sequence, e.g., GAL4 activation domain, (2) a second fusion protein comprising KIAA0796 or a fragment thereof and a second transcriptional regulatory protein sequence, e.g., GAL4 DNA-binding domain, and (3) a reporter gene, e.g., β-galactosidase, which is transcribed when an intermolecular complex comprising the first fusion protein and the second fusion protein is formed. Parallel reactions are performed in the absence of a test agent as the control and in the presence of the test agent. A functional MAPKAP-K3/KIAA0796 complex is detected by detecting the amount of reporter gene expressed. If the amount of reporter gene expression in the presence of the test agent is different than the amount of reporter gene expression in the absence of the test agent, the test agent is a modulator of the interaction of MAPKAP-K3 and KIAA0796.

Candidate agents for modulating the interaction of each of the protein complexes set forth in Tables 2-11 are screened in vivo in a similar manner.

While the invention has been disclosed in this patent application by reference to the details of preferred embodiments of the invention, it is to be understood that the disclosure is intended in an illustrative rather than in a limiting sense, as it is contemplated that modifications will readily occur to those skilled in the art, within the spirit of the invention and the scope of the appended claims.

### LIST OF REFERENCES

Altschul, S. F. et al. (1990). Basic local alignment search tool. J. Mol. Biol. 215:403-410.

Ausubel F M, et al. (1992). Current Protocols in Molecular Biology and supplements, J. Wiley and Sons, New York.

Altschul, S. F. et al. (1997). Gapped BLAST and PSI-BLAST: a new generation of protein database search programs. Nucl. Acids Res. 25:3389-3402.

Apel, E. D. et al. (2000). Syne-1, a dystrophin- and Klarsicht-related protein associated with synaptic nuclei at the neuromuscular junction. J Biol Chem. 275:31986-95.

Aronheim et al., 1997. Isolation of an AP-1 repressor by a novel method for detecting protein-protein interactions. Mol. Cell. Biol. 17:3094-3102.

Bartel, P. L. and Fields, S. (1997). The Yeast Two-Hybrid System. New York: Oxford University Press.

Bartel, P. L. et al. (1993). “Using the 2-hybrid system to detect protein-protein interactions.” In: Cellular Interactions in Development: A Practical Approach, Oxford University Press, pp. 153-179.

Bartel, P. L. et al. (1996). A protein linkage map of Escherichia coli bacteriophage T7. Nat Genet 12:72-77.

Bilbe, G. et al. (1992). Restin: a novel intermediate filament-associated protein highly expressed in the Reed-Stemberg cells of Hodgkin's disease. EMBO J. 11:2103-13.

Cheng, M. et al. (1996). ERK3 is a constitutively nuclear protein kinase. J. Biol. Chem. 271:8951-8.

Chevray, P. M. and Nathans, D. N. (1992). Protein interaction cloning in yeast: identification of mammalian proteins that interact with the leucine zipper of Jun. Proc. Natl. Acad. Sci. USA 89:5789-5793.

Claeskens, A. et al. (2000). Hevin is down-regulated in many cancers and is a negative regulator of cell growth and proliferation. Br. J. Cancer 82:1123-30.

Fields, S. and Song, O-K. (1989). A novel genetic system to detect protein-protein interactions. Nature 340:245-246.

Fritzler, M. J. et al. (1993). Molecular characterization of two human autoantigens: unique cDNAs encoding 95- and 160-kD proteins of a putative family in the Golgi complex. J Exp Med. 178:49-62.

Girard, J. P. and Springer, T. A. (1995). Cloning from purified high endothelial venule cells of hevin, a close relative of the antiadhesive extracellular matrix protein SPARC. Immunity 2:113-23.

Girard, J. P. and Springer, T. A. (1996). Modulation of endothelial cell adhesion by hevin, an acidic protein associated with high endothelial venules. J Biol. Chem. 271:4511-7.

Grammer, A. C. et al. (1998). TNF receptor-associated factor-3 signaling mediates activation of p38 and Jun N-terminal kinase, cytokine secretion, and Ig production following ligation of CD40 on human B cells. J Immunol. 161:1183-93.

Grum, V. L. et al. (1999). Structures of two repeats of spectrin suggest models of flexibility. Cell 98:523-35.

Gueydan, C. et al. (1999). Identification of TIAR as a protein binding to the translational regulatory AU-rich element of tumor necrosis factor alpha mRNA. J. Biol Chem. 274:2322-6.

Harris, V. K. et al. (2000). Mitogen-induced expression of the fibroblast growth factor-binding protein is transcriptionally repressed through a non-canonical E-box element. J Biol Chem. 275:28539-48.

Harlow et al. (1988). Antibodies: A Laboratory Manual (Cold Spring Harbor Laboratory, Cold Spring Harbor, N.Y.).

Heckert, L. L. et al. (2000). The USF proteins regulate transcription of the follicle-stimulating hormone receptor but are insufficient for cell-specific expression. Mol Endocrinol. 14:1836-48.

Hou, J. et al. (1994). Molecular cloning and expression of the gene for a major leucine-rich protein from human hepatoblastoma cells (HepG2). In Vitro Cell. Dev. Biol. Anim. 30A:111-4.

Ismail, P. M. et al. (1999). Loss of USF transcriptional activity in breast cancer cell lines. Oncogene. 18:5582-91.

Jaiswal, A. S. and Narayan, S. (2001). Upstream stimulating factor-1 (USF1) and USF2 bind to and activate the promoter of the adenomatous polyposis coli (APC) tumor suppressor gene. J Cell Biochem. 81:262-77.

Kawasaki, H. et al. (1997). Activation and involvement of p38 mitogen-activated protein kinase in glutamate-induced apoptosis in rat cerebellar granule cells. J Biol Chem. 272:18518-21.

Kirchner, J. et al. (1999). J. Biol Chem. 380:915-21.

Kohler, G. and Milstein, C. (1975). Continuous cultures of fused cells secreting antibody of predefined specificity. Nature 256:495-497.

Kraemer, F. B. et al. (1993). Detection of hormone-sensitive lipase in various tissues. I. Expression of an HSL/bacterial fusion protein and generation of anti-HSL antibodies. J. Lipid Res. 34:663-672.

Leanna, C. A. and Hannink, M. (1996). The reverse two-hybrid system: a genetic scheme for selection against specific protein/protein interactions. Nucl. Acids Res. 24:3341-3347.

Lee, J. C. et al. (1994). A protein kinase involved in the regulation of inflammatory cytokine biosynthesis. Nature 372:739-46.

Lee, J. C. et al. (1996). Role of CSB/p38/RK stress response kinase in LPS and cytokine signaling mechanisms. J Leukoc Biol. 59:152-7.

Luo, X. and Sawadogo, M. (1996). Antiproliferative properties of the USF family of helix-loop-helix transcription factors. Proc Natl Acad Sci USA 93:308-13.

Mach, F. et al. (1998). Reduction of atherosclerosis in mice by inhibition of CD40 signalling. Nature 394:200-3.

Mancini, M. et al. (2000). Caspase-2 is localized at the Golgi complex and cleaves golgin-160 during apoptosis. J Cell Biol. 149:603-12.

Mansfield, P. J. et al. (2000). Regulation of polymorphonuclear leukocyte phagocytosis by myosin light chain kinase after activation of mitogen-activated protein kinase. Blood 95:2407-12.

Miyazawa, K. et al. (1998). Regulation of interleukin-1 beta-induced interleukin-6 gene expression in human fibroblast-like synoviocytes by p38 mitogen-activated protein kinase. J Biol Chem. 273:24832-8.

Moriuchi, M. et al. (1999). USF/c-Myc enhances, while Yin-Yang 1 suppresses, the promoter activity of CXCR4, a coreceptor for HIV-1 entry. J Immunol. 162:5986-92.

Nemoto, S. et al. (1998). Opposing effects of Jun kinase and p38 mitogen-activated protein kinases on cardiomyocyte hypertrophy. Mol Cell Biol. 18:3518-26.

Pierre, P. et al. (1992). CLIP-170 links endocytic vesicles to microtubules. Cell. Cell 70:887-900.

Raingeaud, J. et al. (1995). Pro-inflammatory cytokines and environmental stress cause p38 mitogen-activated protein kinase activation by dual phosphorylation on tyrosine and threonine. J Biol Chem. 270:7420-6.

Sluss, H. K. et al. (1994). Signal transduction by tumor necrosis factor mediated by JNK protein kinases. Mol Cell Biol. 14:8376-84.

Sugimoto, M. et al. (1999). Regulation of CDK4 activity by a novel CDK4-binding protein, p34(SEI-1). Genes Dev. 13:3027-33.

Valetti, C. et al. (1999). Role of dynactin in endocytic traffic: effects of dynamitin overexpression and colocalization with CLIP-170. Mol. Biol. Cell. 10:4107-20.

Wang, Y. et al. (1998). Cardiac muscle cell hypertrophy and apoptosis induced by distinct members of the p38 mitogen-activated protein kinase family. J Biol Chem. 273:2161-8.

Watanabe, H. et al. (2001). Myosin light-chain kinase regulates endothelial calcium entry and endothelium-dependent vasodilation. FASEB J. 15:282-4.

Wetmur, J. G. and Davidson, N. (1968). “Kinetics of renaturation of DNA.” J. Mol. Biol. 31:349-370.

Wigle, J. T. et al. (1999). Prox1 function is required for the development of the murine lymphatic system. Cell 98:769-78.

Zhao, X. et al. (2001). TNF-alpha stimulates caspase-3 activation and apoptotic cell death in primary septo-hippocampal cultures. J Neurosci Res. 2001 64:121-31.

PCT Published Application No. WO 97/27296

PCT Published Application No. WO 99/65939

U.S. Pat. No. 5,622,852

U.S. Pat. No. 5,773,218

What is claimed is:

1. An isolated protein complex comprising two proteins, the protein complex selected from the group consisting of
(a) a complex set forth in Table 1;
(b) a complex set forth in Table 2;
(c) a complex set forth in Table 3;
(d) a complex set forth in Table 4;
(e) a complex set forth in Table 5;
(f) a complex set forth in Table 6;
(g) a complex set forth in Table 7;
(h) a complex set forth in Table 8;
(i) a complex set forth in Table 9;
(h) a complex set forth in Table 10; and
(k) a complex set forth in Table 11.

2. The protein complex of claim 1, wherein said protein complex comprises complete proteins.

3. The protein complex of claim 1, wherein said protein complex comprises a fragment of one protein and a complete protein of anther protein.

4. The protein complex of claim 1, wherein said protein complex comprises fragments of proteins.

5. An isolated antibody selectively immunoreactive with the protein complex of claim 1.

6. The antibody of claim 5, wherein said antibody is a monoclonal antibody.

7. A method for diagnosing a physiological disorder in an animal, which comprises assaying for:
(a) whether a protein complex set forth in any one of Tables 1-11 is present in a tissue extract;
(b) the ability of proteins to form a protein complex set forth in any one of Tables 1-11; and
(c) a mutation in a gene encoding a protein of a protein complex set forth in any one of Tables 1-11.

8. The method of claim 7, wherein said animal is a human.

9. The method of claim 7, wherein the diagnosis is for a predisposition to said physiological disorder.

10. The method of claim 7, wherein the diagnosis is for the existence of said physiological disorder.

11. The method of claim 7, wherein said assay comprises a yeast two-hybrid assay.

12. The method of claim 7, wherein said assay comprises measuring in vitro a complex formed by combining the proteins of the protein complex, said proteins isolated from said animal.

13. The method of claim 12, wherein said complex is measured by binding with an antibody specific for said complex.

14. The method of claim 7, wherein said assay comprises mixing an antibody specific for said protein complex with a tissue extract from said animal and measuring the binding of said antibody.

15. A method for determining whether a mutation in a gene encoding one of the proteins of a protein complex set forth in any one of Tables 1-11 is useful for diagnosing a physiological disorder, which comprises assaying for the ability of said protein with said mutation to form a complex with the other protein of said protein complex, wherein an inability to form said complex is indicative of said mutation being useful for diagnosing a physiological disorder.

16. The method of claim 15, wherein said gene is an animal gene.

17. The method of claim 16, wherein said animal is a human.

18. The method of claim 15, wherein the diagnosis is for a predisposition to a physiological disorder.

19. The method of claim 15, wherein the diagnosis is for the existence of a physiological disorder.

20. The method of claim 15, wherein said assay comprises a yeast two-hybrid assay.

21. The method of claim 15, wherein said assay comprises measuring in vitro a complex formed by combining the proteins of the protein complex, said proteins isolated from an animal.

22. The method of claim 21, wherein said animal is a human.

23. The method of claim 21, wherein said complex is measured by binding with an antibody specific for said complex.

24. A method for screening for drug candidates capable of modulating the interaction of the proteins of a protein complex set forth in any one of Tables 1-11, which comprises:
(a) combining the proteins of said protein complex in the presence of a drug to form a first complex;
(b) combining the proteins in the absence of said drug to form a second complex;
(c) measuring the amount of said first complex and said second complex; and
(d) comparing the amount of said first complex with the amount of said second complex,
wherein if the amount of said first complex is greater than, or less than the amount of said second complex, then the drug is a drug candidate for modulating the interaction of the proteins of said protein complex..

25. The method of claim 24, wherein said screening is an in vitro screening.

26. The method of claim 24, wherein said complex is measured by binding with an antibody specific for said protein complexes.

27. The method of claim 24, wherein if the amount of said first complex is greater than the amount of said second complex, then said drug is a drug candidate for promoting the interaction of said proteins.

28. The method of claim 24, wherein if the amount of said first complex is less than the amount of said second complex, then said drug is a drug candidate for inhibiting the interaction of said proteins.

29. A non-human animal model for a physiological disorder wherein the genome of said animal or an ancestor thereof has been modified such that the formation of a protein complex set forth in any one of Tables 1-11 has been altered.

30. The non-human animal model of claim 29, wherein the formation of said protein complex has been altered as a result of:
(a) over-expression of at least one of the proteins of said protein complex;
(b) replacement of a gene for at least one of the proteins of said protein complex with a gene from a second animal and expression of said protein;
(c) expression of a mutant form of at least one of the proteins of said protein complex;
(d) a lack of expression of at least one of the proteins of said protein complex; or
(e) reduced expression of at least one of the proteins of said protein complex.

31. A cell line obtained from the animal model of claim 29.

32. A non-human animal model for a physiological disorder, wherein the biological activity of a protein complex set forth in any one of Tables 1-11 has been altered.

33. The non-human animal model of claim 32, wherein said biological activity has been altered as a result of:
(a) disrupting the formation of said complex; or
(b) disrupting the action of said complex.

34. The non-human animal model of claim 32, wherein the formation of said complex is disrupted by binding an antibody to at least one of the proteins which form said protein complex.

35. The non-human animal model of claim 32, wherein the action of said complex is disrupted by binding an antibody to said complex.

36. The non-human animal model of claim 32, wherein the formation of said complex is disrupted by binding a small molecule to at least one of the proteins which form said protein complex.

37. The non-human animal model of claim 32, wherein the action of said complex is disrupted by binding a small molecule to said complex.

38. A cell in which the genome of cells of said cell line has been modified to produce at least one protein complex set forth in any one of Tables 1-11.

39. A cell line in which the genome of the cells of said cell line has been modified to eliminate at least one protein of a protein complex set forth in any one of Tables 1-11.

40. A method of screening for drug candidates useful in treating a physiological disorder which comprises the steps of:
(a) measuring the activity of a protein selected from the proteins set forth in Tables 1-11 in the presence of a drug,
(b) measuring the activity of said protein in the absence of said drug, and
(c) comparing the activity measured in steps (1) and (2),
wherein if there is a difference in activity, then said drug is a drug candidate for treating said physiological disorder.
